(19) United States
(12) Patent Application Publication
(10) Pub. No.: US 2009/0028931 A1
(43) Pub. Date: Jan. 29, 2009

(54) Liposomal compositions for parenteral delivery of agents

(75) Inventors: Ellen Wasan, Richmond (CA); Marcel Bally, Bowen Island (CA); Sebastian Cogswell, Vancouver (CA); Stuart Berger, Toronto (CA)

Correspondence Address: RONALD I. EISENSTEIN;NIXON PEABODY LLP, 100 SUMMER STREET, BOSTON, MA, 02110, US

(73) Assignees: BC Cancer Agency, Vancouver, BC (CA); University Health Network, Toronto, ON (CA)

(21) Appl. No.: 11/883,255

(22) PCT Filed: Jan. 30, 2006
(86) PCT No.: PCT/CA06/00114

§ 371 Date: Jun. 5, 2008

Related U.S. Application Data
(60) Provisional application No. 60/647,419, filed on Jan. 28, 2005.

(51) Int. Cl. A61K 9/127; A61K 31/4164; A61P 35/00; A61P 31/10

(52) U.S. Cl. 424/450; 514/399

(57) ABSTRACT

The invention provides methods and compositions for loading an agent, such as econazole, onto a liposome for parental delivery. The loading of the agent into a liposome comprises combining the agent with a micelle-forming compound to form a micelle including the agent, where the agent is releasable from the micelle-forming compound, and adding the micelle to the liposome, where the micelle combines with the liposome such that the agent is loaded into the liposome to form a loaded liposome. The methods are suitable for the loading of poorly soluble agents onto liposome.

### CROSS-REFERENCE TO RELATED APPLICATIONS

This application claims the benefit of U.S. provisional application No. 60/647,419, filed Jan. 28, 2005, which is hereby incorporated by reference.

### FIELD OF THE INVENTION

The invention is, in general, in the field of drug delivery. More specifically, the invention provides methods and compositions for parenteral delivery of an agent, using a liposome delivery vehicle.

### BACKGROUND OF THE INVENTION

Liposomes are microscopic particles that are made up of one or more lipid bilayers enclosing an internal compartment. Liposomes have been widely studied and used as carriers for a variety of agents such as drugs, cosmetics, diagnostic reagents, and genetic material. Since liposomes consist of non-toxic lipids, they generally have low toxicity and therefore are useful in a variety of pharmaceutical applications. In particular, liposomes are useful for increasing the circulation lifetime of agents that have a short half-life in the bloodstream. Liposome-encapsulated agents often have biodistributions and toxicities which differ greatly from those of free agent. For specific in vivo delivery, the sizes, charges and surface properties of these carriers can be changed by varying the preparation methods and by tailoring the lipid makeup of the carrier. For instance, liposomes may be made to release an agent more quickly by decreasing the acyl chain length of a lipid making up the carrier.

Agents can be encapsulated in liposomes using a variety of methods and include membrane partitioning, passive encapsulation and active encapsulation. Agents that have hydrophobic attributes can intercalate into the lipid bilayer and this can be achieved by adding the agent during the liposome manufacturing process or by adding the agent to pre-formed liposomes. Agent encapsulation is often limited due to the ability of the liposome membrane to stably incorporate the agent. In addition the agent may adversely impact the physical properties of the liposomes. This method is also limited because the associated agent can rapidly transfer out of the membrane.

Passive loading involves the use of water soluble agents which are added to liposomes during the manufacturing process. Some of the added agent will be encapsulated in the aqueous core of the liposomes and free agent (agent that has not been trapped within the liposome) can be removed by several standard separation methods. This procedure typically results in low trapping efficiencies and low agent to lipid ratios and is, therefore, not ideal.

Active loading techniques have been used to achieve high concentrations of agent in liposomes. Active loading involves the creation of pH gradients (ΔpH) or metal ion gradients (ΔM2+) across the liposomal bilayer. For example, a ΔpH generated by preparing liposomes in citrate buffer pH 4.0, followed by exchange of external buffer with buffered-saline pH 7.5, can promote the liposomal accumulation of weakly basic agent. The neutral form of the agent passively diffuses across the lipid bilayer and becomes trapped upon protonation in the low pH environment of the liposome interior. This process can result in >98% agent encapsulation and high agent-to-lipid ratios (e.g. vinorelbine, doxorubicin, vincristine, daunoruibicin, mitcxantrone, to name a few). However, successful loading and retention using a transmembrane pH gradient is realized while the internal pH of the liposome is maintained. Since the pH gradient can dissipate following agent loading and since maintenance of the pH gradient is critical to achieve optimal agent retention, clinical formulation of pH gradient loaded agents requires the generation of a pH gradient across the liposomes just prior to agent loading or the use of formulations that maintain the pH gradient effectively after loading (e.g. use of strong buffers or ionophores that engender pH gradient formation). A second disadvantage of this method results from instability of lipid, and some agents, at acidic pH which decreases the long-term storage potential of the agent loaded liposomes. Freezing of liposomal formulations slows the rate of hydrolysis but conventional liposomal formulations often aggregate and leak contents upon thawing unless appropriately selected cryoprotectants are used.

Agentloading via ΔM2+ follows a process analogous to the pH gradient process, with agent accumulation being driven by metal ion-complexation (e.g. doxorubicin-Mn2+). Agentloading efficiencies are comparable to those described for the ΔpH process. However, loading efficiency is dependent on the choice of metal ion and agent.

### SUMMARY OF THE INVENTION

The invention provides methods for loading an agent onto a liposome for parenteral delivery, compositions prepared using the methods, and uses thereof.

In one aspect, the invention provides a method for loading an agent into a liposome by combining the agent with a micelle-forming compound to form a micelle including the agent, where the agent is releasable from the micelle-forming compound, and adding the micelle to the liposome, where the micelle combines with the liposome such that the agent is loaded into the liposome to form a loaded liposome.

In alternative embodiments, the micelle may combine with the lipid bilayer of the liposome; the micelle-forming compound may include a hydrophilic or amphipathic moiety such as a PEG-lipid conjugate (e.g., DSPE-PEG2000)

In alternative embodiments, the agent may be dissolved in a solvent, such as ethanol. In alternative embodiments, the agent may be a compound that is poorly soluble. In alternative embodiments, the agent may be a therapeutic agent (e.g., econazole or an anticancer agent or an antifungal agent).

In alternative embodiments, the loaded liposome may be about 100 nm to about 200 nm in diameter. In alternative embodiments, the loaded liposome may be a unilamellar liposome. In alternative embodiments, the loaded liposome may include one or more of a lipid selected from DMPC or DPPC. In alternative embodiments, the loaded liposome may include a targeting agent.

In alternative aspects, the invention provides a composition produced by a method of the invention. In alternative embodiments, the composition may further include a pharmaceutically acceptable carrier.

In alternative aspects, the invention provides a liposomal composition including econazole, where the composition is formulated for parenteral delivery. In alternative embodiments, the composition may further include a lipid selected from DMPC or DPPC. In alternative embodiments, the composition may further include DSPE-PEG2000.

In alternative aspects, the invention provides a method of treating a cancer or a fungal infection by administering a composition of the invention to a subject in need thereof. In alternative aspects, the invention provides the use of a composition of the invention for preparation of a medicament for treating a cancer or a fungal infection in a subject in need thereof. In alternative aspects, the invention provides a method of delivering a therapeutic agent to a cell in a subject in need thereof by administering the composition of the invention to the subject.

In alternative aspects, the invention provides a method for selecting a liposome composition having a desired loading or retention property for an agent, by preparing a first liposome composition by combining a vesicle-forming lipid with the agent under conditions suitable for forming a liposome such that the agent is loaded into the liposome; preparing a second liposome composition by combining the agent with a micelle-forming compound to form a micelle including the therapeutic agent, where the agent is releasable from the micelle-forming compound; adding the micelle to a liposome, where the micelle combines with the liposome such that the agent is loaded into the liposome; determining the amount of agent loaded onto the liposome or retained in the liposome in the first liposome composition and the second liposome composition, where a greater amount of agent loaded onto the liposome or retained in the liposome in the second liposome composition indicates a liposome composition having a desired loading or retention property in vitro or in vivo for the agent.

In alternative aspects, the invention provides a kit for preparing a loaded liposome including a first container including a therapeutic agent solubilized in a micelie and a second container including a liposome of the desired composition, together with instructions for combining the contents of the first and second containers to prepare a loaded liposome.

In alternative aspects, the invention provides a kit for preparing a loaded liposome including a first container including a therapeutic agent; a second container including a micelle-forming compound; and a third container including a liposome of the desired composition, together with instructions for combining the contents of the first and second containers to form a micelle including the therapeutic agent, and for combining the micelle with the contents of the third container to prepare a loaded liposome. In alternative embodiments, the therapeutic agent may be econazole; the micelle may include DSPE-PEG2000; and/or the liposome may include a lipid selected from DMPC or DPPC.

### BRIEF DESCRIPTION OF THE DRAWINGS

FIG. 1 depicts the chemical structure of econazole: [(dichloro-2,4 phenyl)-2(chloro-4 benzoyloxy)-2 ethyl]-1 imidazole nitrate.

FIG. 2 is a graph demonstrating the efficacy of econazole as a direct injection in MCF7 breast cancer xenografts in mice. Symbols: squares: DMSO vehicle control; circles: econazole 50 mg/mL in DMSO; triangles econazole 100 mg/mL in DMSO.

FIGS. 3A-B are schematic diagrams of the formulations. Symbols: curved lines: DSPE-PEG. Triangles: econazole. A. DSPE-PEG micelles added externally to liposomes containing econazole in the bilayer; B. DSPE-PEG/econazole micelles added to the outer leaflet of preformed liposomes.

FIGS. 4A-B are graphs demonstrating the drug to lipid ratio for during micelle exchange at 50° C. A: DMPC/DSPE-PEG (95:5 mol:mol); B: DPPC/DSPE-PEG (95:5 mol:mol). Diamonds (--⋄--) represent data for thin film/extrusion method of incorporating econazole into the liposomes and squares (-▪-) represent data for liposomal econazole prepared by the micelle exchange method. Data represent mean±SD for 3 separate experiments within which each measurement was also performed in triplicate.

FIGS. 5A-B are bar graphs demonstrating the stability of liposomal econazole after 3, 10 or 20 days at 4° C. in HEPES buffered 150 mM NaCl (pH 7.2). A: DMPC/DSPE-PEG (95:5 mol:mol); A: DPPC/DSPE-PEG (95:5 mol:mol). Black bars: thin film/extrusion method of incorporating econazole into the liposomes; White bars: micelle-loading method. Data represent mean±SD (n=3).

FIGS. 6A-D are graphs demonstrating the stability of micelle-loaded liposomal econazole. Liposomal econazole was incubated in HEPES-buffered saline (pH 7.2) or human plasma for 30 min at 37° C., followed by fractionation by gel filtration chromatography into liposome, micelle and protein-containing fractions. A and B represent the fractional distribution of DMPC/DSPE-PEG (95:5 mol:mol) formulations; C and D represent DPPC/DSPE-PEG (95:5 mol:mol) formulations. A and C show liposome components and B and D show econazole and protein fractional distribution. Black symbols represent samples that were incubated in buffer, while open symbols represent samples that were incubated in plasma. Symbols: Circles: liposomal lipid; squares: DSPE-PEG₂₀₀₀, triangles: econazole, diamonds: total protein (shown on B only for clarity). Data are mean±SD, n=3 separate liposome preparations)

FIGS. 7A-B are graphs demonstrating the plasma elimination profile of liposomal econazole. Points represent 6 mice per timepoint (mean econazole concentration±SD). A: Econazole elimination from plasma. B: drug to lipid ratio (w/w) vs. time

FIGS. 8A-B are graphs demonstrating the efficacy of liposomal econazole against MCF-7 tumours grown as xenografts in immunocompromised Rag2M mice. A: Treatment with liposomal econazole composed of DPPC/DSPE-PEG (95:5 mol/mol, micelle-loaded method) at 50 mg/kg or empty liposome vehicle control on days 17, 20, 22, 24, 27 and 29 (Indicated as ↑ on graph), starting when tumours were approximately 50 mm³. Data represent mean±SEM (n=6 for vehicle controls and untreated controls, and n=5 for liposomal econazole treatment group). B: Data represents mean±SEM for each treatment group (L-Econ: liposomal econazole; VC: vehicle control; UC: untreated control) for days 41-51 to illustrate the trend in controlling tumour growth for the liposomal econazole treatment group.

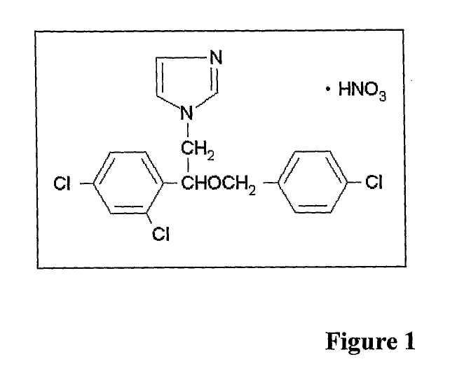

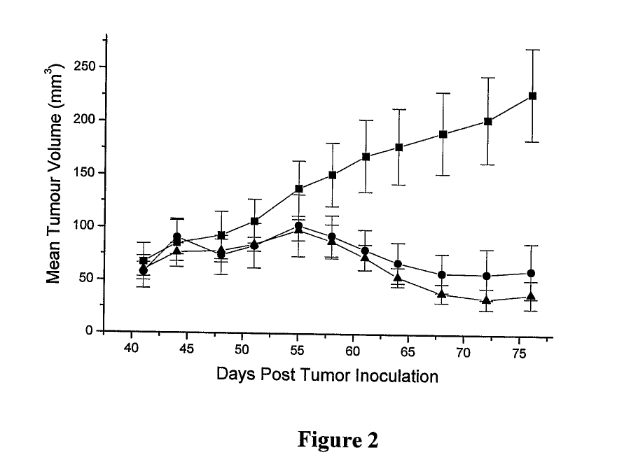

### DETAILED DESCRIPTION OF THE INVENTION

The present invention provides, in part, liposomal compositions for parenteral delivery of an agent (e.g., a therapeutic agent), and methods of preparation thereof. In some embodiments, the invention provides methods for increasing the concentration of poorly soluble compounds (e.g., hydrophobic compounds) that can be achieved in liposomes. In some embodiments, the invention provides methods for increased incorporation of poorly soluble compounds into liposomes. In some embodiments, such methods may: reduce the amount of a solvent required to solubilize a poorly soluble compound; or may extend the stability of liposomes containing a poorly soluble compound in the bloodstream of a subject; or may extend the stability of liposomes containing a poorly soluble compound during storage; or may increase the retention of a poorly soluble compound within a liposome during storage or in circulation in the bloodstream of a subject; or may otherwise improve the properties of a liposome containing a poorly soluble compound generally, either in vitro or in vivo. In some embodiments, use of a micelle as a means to solubilize a poorly soluble compound to be incorporated into a liposome increases the amount of that compound that can be stably incorporated into the liposome bilayer.

### Liposomes

The term “liposome” as used herein means a vesicle including one or more concentrically ordered lipid bilayer(s) encapsulating an aqueous phase, when in an aqueous environment. Formation of such vesicles requires the presence of “vesicle-forming lipids” which are defined herein as amphipathic lipids capable of either forming or being incorporated into a bilayer structure. The term includes lipids that are capable of forming a bilayer by themselves or when in combination with another lipid or lipids. An amphipathic lipid is incorporated into a lipid bilayer by having its hydrophobic moiety in contact with the interior, hydrophobic region of the bilayer membrane and its polar head moiety oriented towards an outer, polar surface of the membrane. Hydrophilicity arises from the presence of functional groups such as hydroxyl, phosphate, carboxyl, sulfate, amino or sulfhydryl groups. Hydrophobicity results from the presence of a long chain of aliphatic hydrocarbon groups.

Liposomes can be categorized into multilamellar vesicles, multivesicular liposomes, unilamellar vesicles and giant liposomes. Multilamellar liposomes (also known as multilamellar vesicles or “MLV”) contain multiple concentric bilayers within each liposome particle, resembling the “layers of an onion”. Multivesicular liposomes consist of lipid membranes enclosing multiple non-concentric aqueous chambers. Unilamellar liposomes enclose a single internal aqueous compartment. Single bilayer (or substantially single bilayer) liposomes include small unilamellar vesicles (SUV) and large unilamellar vesicles (LUV). LUVs and SUVs range in size from about 50 to 500 nm and 20 to 50 nm respectively. Giant liposomes typically range in size from 5000 nm to 50,000 nm and are used mainly for studying mechanochemical and interactive features of lipid bilayer vesicles in vitro (Needham et al., Colloids and Surfaces B: Biointerfaces (2000) 18: 183-195).

Any suitable vesicle-forming lipid may be utilized in the practice of this invention as judged by one of skill in the art. This includes phospholipids such as phosphatidylcholine (PC), phosphatidylglycerol (PG), phosphatidylinositol (PI), phosphatidic acid (PA), phosphatidyethanolamine (PE) and phosphatidylserine (PS); sterols such as cholesterol; glycolipids; sphingolipids such as sphingosine, ceramides, sphingomyelin, and glycosphingolipids (such as cerebrosides and gangliosides). Suitable phospholipids may include one or two acyl chains having any number of carbon atoms, between about 6 to about 24 carbon atoms, selected independently of one another and with varying degrees of unsaturation. Thus, combinations of phospholipid of different species and different chain lengths in varying ratios may be selected. Mixtures of lipids in suitable ratios, as judged by one of skill in the art, may also be used.

Liposomes for use in the present invention may be generated using a variety of conventional techniques. These techniques include: the ether injection method (Deamer et al., Acad. Sci. [1978] 308:250); the surfactant method (Brunner et al., [1976] Biochim. Biophys. Acta, 455:322); the Ca²⁺ fusion method (Paphadjopoulos et al., [1975] Biochim. Biophys. Acta, 394:483); the freeze-thaw method (Pick et al., [1981] Arach. Biochim. Biophys., 212:186); the reverse-phase evaporation method (Szoka et al., [1980] Biochim. Biophys. Acta, 601:559); the ultrasonic treatment method (Huang et al. [1969] Biochemistry, 8:344); the ethanol injection method (Kremer et al. [1977] Biochemistry, 16:3932); the extrusion method (Hope et al., [1985] Biochimica et Biophysica Acta, 812:55); the French press method (Barenholz et al., [1979] FEBS Lett., 99:210); or any other technique described herein or known in the art.

Different techniques may be appropriate depending on the type of liposome desired. For example, small unilamellar vesicles (SUVs) can be prepared by the ultrasonic treatment method, the ethanol injection method, or the French press method, while multilamellar vesicles (MLVs) can be prepared by the reverse-phase evaporation method or by the simple addition of water to a lipid film followed by dispersal by mechanical agitation (Bangham et al., [1965] J. Mol. Biol. 13:238-252). LUVs may be prepared by the ether injection method, the surfactant method, the Ca2+ fusion method, the freeze-thaw method, the reverse-phase evaporation method, the French press method or the extrusion method. In some embodiments, LUVs are prepared according to the extrusion method. The extrusion method involves first combining lipids in chloroform to give a desired molar ratio. A lipid marker may optionally be added to the lipid preparation. The resulting mixture is dried under a stream of nitrogen gas and placed in a vacuum pump until the solvent is substantially removed. The samples are then hydrated in an appropriate buffer or mixture of therapeutic agent or agents. The mixture is then passed through an extrusion apparatus (e.g. Extruder, Northern Lipids, Vancouver, BC) to obtain liposomes of a defined size. Average liposome size can be determined by quasi-elastic light scattering or photon correlation spectroscopy or dynamic light scattering or various electron microscopy techniques (such as negative staining transmission electron microscopy, freeze fracture electron microscopy or cryo-transmission electron microscopy). If desired, the resulting liposomes may be run down a Sephadex™ G50 column or similar size exclusion chromatography column equilibrated with an appropriate buffer in order to remove unencapsulated drug or to create an ion gradient by exchange of the exterior buffer. Subsequent to generation of an ion gradient, LUVs may encapsulate therapeutic agents as set forth herein.

In some aspects, liposomes are prepared to be “cholesterol free”, meaning that such lipid-based vehicles contain “substantially no cholesterol,” or contain “essentially no cholesterol.” The term “cholesterol-free” as used herein with reference to a liposome means that the liposome is prepared in the absence of cholesterol, or contains substantially no cholesterol, or that the vehicle contains essentially no cholesterol. The term “substantially no cholesterol” allows for the presence of an amount of cholesterol that is insufficient to significantly alter the phase transition characteristics of the liposome (typically less than 20 mol % cholesterol). 20 mol % or more of cholesterol broadens the range of temperatures at which phase transition occurs, with phase transition disappearing at higher cholesterol levels. A liposome having substantially no cholesterol may have about 15 or less, or about 10 or less mol % cholesterol. The term “essentially no cholesterol” means about 5 or less mol %, or about 2 or less mol %, or about 1 or less mol % cholesterol. In some embodiments, no cholesterol will be present or added when preparing “cholesterol-free” liposomes. The presence or absence of cholesterol will influence the ability of the micelle-solubilized compound that can be stably incorporated into the liposome bilayer and will influence retention of that compound after incorporation.

Liposomes may range from any value between about 50 nm to about 1 um in diameter. For example, liposomes in a liposomal composition according to the invention may range from any value between about 100 to about 140 nm in diameter. In some embodiments, liposomes in a liposomal composition according to the invention may be less than about 200 nm in diameter, or less than about 160 nm in diameter, or less than about 140 nm in diameter. In some embodiments, liposomes in a liposomal composition according to the invention may be substantially uniform in size, for example, 10% to 100%, or more generally at least 10%, 20%, 30%, 40%, 50, 55% or 60%, or at least 65%, 75%, 80%, 85%, 90%, or 95%, or as much as 96%, 97%, 98%, 99%, or 100% of the liposomes in the liposomal composition may be between the size values indicated herein. Liposomes may be sized by extrusion through a filter (e.g. a polycarbonate filter) having pores or passages of the desired diameter.

Liposomes may include a targeting agent (such as a sugar moiety, a cell receptor ligand, an antibody specific to a target cell, such as a cancer cell, etc.) to achieve enhanced targeting to a specific cell population. Targeting agents may be incorporated into the surface of a liposome to optimize binding to target cells.

In some embodiments, liposomes may include a hydrophilic moiety. Grafting a hydrophilic moiety to the surface of liposomes can “sterically stabilize” liposomes thereby maximizing the circulation longevity of the liposome. This results in enhanced blood stability and increased circulation time, reduced uptake into healthy tissues, and increased delivery to disease sites such as solid tumors (see U.S. Pat. Nos. 5,013,556 and 5,593,622; and Patel et al., [1992] Crit Rev Ther Drug Carrier Syst, 9:39). Typically, the hydrophilic moiety is conjugated to a lipid component of the liposome, forming a hydrophilic polymer-lipid conjugate. The term “hydrophilic polymer-lipid conjugate” refers to a lipid, e.g., a vesicle-forming lipid, covalently joined at its polar head moiety to a hydrophilic polymer, and is typically made from a lipid that has a reactive functional group at the polar head moiety in order to attach the polymer. The covalent linkage may be releasable such that the polymer may dissociate from the lipid at for example physiological pH after a variable length of time, such as over several to many hours (Adlakha-Hutcheon et al. [1999] Nat Biotechnol. 17(8):775-9). Suitable reactive functional groups are for example, amino, hydroxyl, carboxyl or formyl groups. The lipid may be any lipid described in the art for use in such conjugates. The lipid may be a phospholipid having one or two acyl chains including between about 6 to about 24 carbon atoms in length with varying degrees of unsaturation.

In some embodiments, the lipid in the conjugate may be a PE, such as of the distearoyl form. The polymer may be a biocompatible polymer characterized by a solubility in water that permits polymer chains to effectively extend away from a liposome surface with sufficient flexibility that produces uniform surface coverage of a liposome. Such a polymer may be a polyalkylether, including polyethylene glycol (PEG), polymethylene glycol, polyhydroxy propylene glycol, polypropylene glycol, polylactic acid, polyglycolic acid, polyacrylic acid and copolymers thereof, as well as those disclosed in U.S. Pat. Nos. 5,013,556 and 5,395,619. The polymer may have an average molecular weight of any value between about 350 and about 10,000 daltons.

In alternative embodiments, the phospholipids may be selected from poly(ethylene glycol) (PEG) modified phospholipids. The average molecular weight of the PEG may be any value between about 500 to about 10,000 Daltons. Combinations of PEG phospholipid of different species and different chain lengths in varying ratios may be selected. Combinations of phospholipids and PEG phospholipids may also be selected. The conjugate may be prepared to include a releasable lipid-polymer linkage such as a peptide, ester, or disulfide linkage. The conjugate may also include a targeting agent. Mixtures of conjugates may be incorporated into liposomes for use in this invention.

In some embodiments, liposomes may include an agent, such as a therapeutic agent, prepared by conventional “active” or “passive” loading methods. For example, a therapeutic agent can be mixed with vesicle-forming lipids and be incorporated within a lipid film, such that when the liposome is generated, the therapeutic agent is incorporated or encapsulated into the liposome. Thus, if the therapeutic agent is substantially hydrophobic, it will be encapsulated in the bilayer of the liposome. Alternatively, if the therapeutic agent is substantially hydrophilic, it will be encapsulated in the aqueous interior of the liposome. The therapeutic agent may be soluble in aqueous buffer or aided with the use of detergents or ethanol. The liposomes can subsequently be purified, for example, through column chromatography or dialysis to remove any unincorporated therapeutic agent.

Liposomes may be prepared and formed in advance i.e., be “pre-formed” liposomes. Pre-formed liposomes may be used to prepare the liposomal formulations according to the invention. Such pre-formed liposomes may include an agent, such as a therapeutic agent, or an agent may be added to pre-formed liposomes prior to preparation of liposomal compositions according to the invention e.g., prior to combination with a micelle containing an agent. In some embodiments, pre-formed liposomes do not include a hydrophilic moiety. Pre-formed liposomes are available from various commercial contract pharmaceutical companies with expertise in the art of preparing liposomes.

### Micelles

The term “micelle” as used herein means a vesicle including a single lipid monolayer encapsulating an aqueous phase. Micelles may be spherical or tubular or wormlike and form spontaneously about the critical micelle concentration (CMC). In general, micelles are in equilibrium with the monomers under a given set of physical conditions such as temperature, ionic environment, concentration, etc.

Formation of a micelle requires the presence of “micelle-forming compounds,” which include amphipathic lipids (e.g., a vesicle-forming lipid as described herein or known in the art), lipoproteins, detergents, non-lipid polymers, or any other compound capable of either forming or being incorporated into a monolayer vesicle structure. Thus, a micelle-forming compound includes compounds that are capable of forming a monolayer by themselves or when in combination with another compound, and may be polymer micelles, block co-polymer micelles, polymer-lipid mixed micelles, or lipid micelles. A micelle-forming compound, in an aqueous environment, generally has a hydrophobic moiety in contact with the interior of the vesicle, and a polar head moiety oriented outwards into the aqueous environment. Hydrophilicity generally arises from the presence of functional groups such as hydroxyl, phosphate, carboxyl, sulfate, amino or sulfhydryl groups. Hydrophobicity generally results from the presence of a long chain of aliphatic hydrocarbon groups.

A micelle may be prepared from lipoproteins or artificial lipoproteins including low density lipoproteins, chylomicrons and high density lipoproteins. Artificial lipoproteins may also comprise lipidized protein with targeting capabilities. Uptake of lipoproteins into cell populations may be facilitated by receptors present on the target cells. For instance, uptake of low density lipoproteins into cancerous cells may be facilitated by LDL receptors present on such cells and uptake of chylomicrons and lactosylated high density lipoproteins into hepatocytes may be facilitated by the remnant receptor and the lactosylated receptor respectively.

Micelles for use in the present invention may be generated using a variety of conventional techniques. These techniques include: simple dispersion by mixing in aqueous or hydroalcoholic media or media containing surfactants or ionic substances; sonication, solvent dispersion or any other technique described herein or known in the art. Different techniques may be appropriate depending on the type of micelle desired and the physicochemical properties of the micelle-forming components, such as solubility, hydrophobicity and behaviour in ionic or surfactant-containing solutions.

Micelles for use in the present invention may range from any value between about 5 nm to about 50 nm in diameter. In some embodiments, micelles may be less than about 50 nm in diameter, or less than about 30 nm in diameter, or less than about 20 nm in diameter.

In some embodiments, micelles for use in the present invention may include a hydrophilic polymer-lipid conjugate, as described herein or known in the art. As indicated herein, the term “hydrophilic polymer-lipid conjugate” refers to a lipid, e.g., a vesicle-forming lipid, covalently joined at its polar head moiety to a hydrophilic polymer, and is typically made from a lipid that has a reactive functional group at the polar head moiety in order to attach the polymer. The covalent linkage may be releasable such that the polymer may dissociate from the lipid at for example physiological pH after a variable length of time, such as over several to many hours (Adlakha-Hutcheon et al. [1999] Nat Biotechnol. 17(8):775-9). Such conjugates may include any compounds known and routinely utilized in the art of sterically stabilized liposome technology and technologies which are useful for increasing circulatory half-life for proteins, including for example polyethylene glycol (PEG), polyvinyl alcohol, polylactic acid, polyglycolic acid, polyvinylpyrrolidone, polyacrylamide, polyglycerol, or synthetic lipids with polymeric head groups. For example, a distearoyl-phosphatidylethanolamine covalently bonded to a PEG alone, or in further combination with phosphatidylcholine (PC), may be used to produce a micelle according to the invention. The molecular weight of the PEG may be any value between about 500 Daltons to about 10,000 Daltons, inclusive, for example, 1000, 2000, 4000, 6000, 8000, etc. The CMC of the hydrophilic polymer-lipid conjugate will be dependent on the molecular weight of the PEG as well as the lipid anchor and the added components used when preparing mixed micelles (e.g. PEG modified distearoyl-phosphatidylethanolamine and PC).

### Methods of Preparing Liposomal Compositions

The invention provides a method of preparing a liposomal composition including an agent or compound (e.g., a therapeutic agent such as econazole, which is used herein as a model compound) by incorporating the agent or compound into a micelle. The micelle may include a PEG-phospholipid, such as DSPE-PEG₂₀₀₀. The micelle is then combined with a liposome, such as a pre-formed liposome, thus incorporating the agent or compound into the liposome. In alternative embodiments, the agent is a poorly soluble compound that can be solubilized in a micelle. In alternative embodiments, liposomal compositions according to this invention are particularly suitable for the delivery of poorly soluble compounds or agents.

Any active agent may be used in the liposomal compositions according to the invention. An “active agent” or “agent” or “compound” as used herein refers to a chemical moiety used in therapy or diagnosis, and includes any natural or synthetic biologically active agent, such as a peptide or polypeptide or analog thereof, a nucleic acid molecule or analog thereof, a small molecule, a prodrug, etc., and for which drug delivery in accordance with this invention is desirable. Thus, an agent includes therapeutic agents and imaging agents. The term “prodrug” as used herein refers to any compound that has less intrinsic activity than the corresponding “drug,” but when administered to a biological system, generates the “drug” substance, either as a result of spontaneous chemical reaction or by enzyme catalyzed or metabolic reaction. Prodrugs include, without limitation, acyl esters, carbonates, phosphates, and urethanes. These groups are exemplary, and not exhaustive, and one skilled in the art could prepare other known varieties of prodrugs.

The agent or compound may be of any class which can be solubilized and incorporated into a micelle that includes micelle forming compounds. In alternative embodiments, the agent is “poorly soluble” in water or buffer, or under physiological conditions. A “poorly soluble” compound or agent is one that exhibits very low solubility, or is insoluble, in an aqueous environment, e.g., in an aqueous buffered solution at concentrations suitable for administration of pharmacologically relevant dosages of said compounds. In some embodiments, the term “poorly soluble” with reference to an active agent in water or buffer or physiological saline means that the active agent has a solubility in the water or buffer of less than about 10 mg/mL. Compound solubility can be measured and defined using standard techniques, for example, as indicated in the The United States Pharmacopoeia/The National Formulary standards and guidelines or other scientific reference manuals such as the Merck Index (Merck Co., Rahway, N.J.), or by any other means known in the art. For example, solubility of poorly soluble compounds can be quantified based on octanol-water partition coefficient (LogP) or hydrophile-lipophile balance (HLB) scale (see for example Schott [1995] J Pharm Sci. 84(10):1215-22) and Schott [1984] J Pharm Sci. 73(6):790-2). In some embodiments, a poorly soluble compound exhibits a LogP of at least 1.5 or more. In some embodiments, a poorly soluble compound is one that is soluble in about 30 to about 10,000 or more parts of water for one part of solute, or from about 100 to about 1000 parts water/part solute, or from about 100 to about 10000 parts water/part solute, or from about 30 to about 100 parts water/part solute. The desired amount of compound to be incorporated into a liposome will depend in part on the potency of the compound where lower concentrations of a compound may be necessary for a potent compound. Poorly soluble compounds include without limitation lipid soluble compounds, hydrophobic compounds, compounds poorly soluble at physiological pH, etc. In one embodiment, a poorly soluble compound is an azole compound, such as econazole.

In some embodiments, the compound may be solubilized in a solvent, such as ethanol or hydroalcoholic solutions of ethanol in aqueous media, prior to incorporation into the micelle. In some embodiments, the final concentration of solvent in the phospholipid-containing liposomes, for example those composed primarily of DPPC, DMPC, DSPC, DOPC or similar compositions, may be limited to a concentration that does not induce significant toxicity when administered to a subject and/or does not disrupt the integrity or performance of the micelle or liposome. For example, for ethanol, the final concentration may be any value between about 1 to about 30% (v/v), although lower or higher values are also contemplated. In some embodiments, the incorporation of poorly soluble compounds into liposomes can be achieved while minimizing solvent concentrations or the presence of bio-incompatible solvents. For compounds to be encapsulated within the liposomal bilayer which are directly soluble in aqueous dispersions of the micelle-forming components, solvents such as ethanol may not be necessary.

A compound or agent may be incorporated into a micelle during preparation of a micelle as described herein or known in the art. In alternative embodiments, the compound or agent is not covalently coupled to, e.g., are releasable from, a micelle forming compound.

The compound-containing micelles are then incorporated into the liposomes. The liposomes may include but are not limited to one or more of the following lipids: DMPC, DPPC or DSPE, and the ratios of the lipids may vary according to embodiments visualized by persons skilled in the art of liposome preparation. In some embodiments, the liposome may be a pre-formed liposome that may or may not contain the therapeutic agent or one or more second or additional agent(s) (e.g., a small molecule, a protein, antibody, or polypeptide or a nucleic acid, e.g., having membrane localization properties such as juxtamembrane localization or transmembrane domains) incorporated or encapsulated in it. The second or additional agent may be loaded into the liposome using conventional loading techniques as described herein or known in the art. Alternatively, or additionally, more than one compound may be loaded into a liposome using the methods of the invention, by for example incorporating one or more micelles containing one or more compounds into the liposome. In an alternative embodiment, small molecules (chemical compounds), proteins, antibodies or peptides or pharmaceutically acceptable salt thereof, may be encapsulated into a liposome by prior solubilization, active loading or passive entrapment and incorporation into a polymer micelles, polymer-lipid mixed micelles or lipid micelles.

Liposomal compositions according to the invention may be stored in any suitable form that may vary according to mode of administration. For example, a liposomal composition may be a liquid at room temperature (e.g., a sterile single-vial liquid), a frozen product, or a dehydrated product (e.g., a powder or a lyophilized cake to be reconstituted prior to use). Different storage forms may be prepared using methods known to a person skilled in the art. For example, a cryoprotectant such as a disaccharide, may be added to a liposomal composition prior to lyophilization to enable storage of a liposomal composition as a dehydrated product.

In alternative embodiments, the compound or agent is releasable from (e.g., not covalently coupled to a vesicle forming lipid or a micelle forming compound) a liposome prepared according to the invention, to facilitate transfer of the compound or agent into a target cell. Thus, a releasable agent is an agent that is capable of transferring out of a liposome according to the invention and exerting its biological action inside, or in the vicinity of, a cell in a subject. In alternative embodiments, the compound or agent is generally stable during storage of a liposomal composition. In alternative embodiments, the compound or agent is generally stable during circulation in the bloodstream of a subject i.e., the compound or agent is not substantially released from the liposome prior to its delivery inside, or in the vicinity of, a cell in a subject.

As described herein, econazole PEG-lipid micelles including DSPE-PEG₂₀₀₀ were added to pre-formed liposomes including DMPC or DPPC. Econazole was rapidly loaded, and remained stably incorporated, into these liposomes.

In alternative aspects, liposomal compositions including an agent, prepared according to the invention (e.g., by combination of a micelle containing the agent with a liposome) may be compared to liposomal compositions containing an agent, prepared using conventional techniques. Such comparisons may be used to select compounds having desired loading or retention properties, such as increased concentration or loading in the liposomal compositions according to the invention, or such as greater stability of the liposomal compositions according to the invention in storage or in circulation in the bloodstream of a subject, or such as greater retention of the agent in the liposomal compositions according to the invention. By increased or greater concentration, or stability, or storage, is meant an increase of any value between 10% and 90%, or of any value between 30% and 60%, or over 100%, such as two fold, or five-fold, or greater than ten-fold, in a liposomal composition prepared according to the invention when compared with a liposomal composition prepared using convention techniques, such as thin-film extrusion.

### Therapeutic Indications and Agents

Liposomal compositions according to this invention may be used for delivery of a therapeutic agent, for example a poorly soluble therapeutic agent, for treatment of a variety of diseases and conditions in a subject in need thereof, or for bringing about a desired biological effect such as an immune response in such a subject. Such diseases and conditions include those that would benefit from liposomes which increase retention or stability of the therapeutic agent in storage or in circulation in a subject, enabling therapeutic drug interventions with superior ADMET (absorption, distribution, metabolism, excretion and toxicity) properties. Examples of therapeutic uses of the compositions of the present invention include treating cancer, treating cardiovascular diseases such as hypertension, cardiac arrhythmia and restenosis, treating bacterial, viral, fungal or parasitic infections, treating and/or preventing diseases through the use of the compositions of the present inventions as vaccines, treating inflammation or treating autoimunune diseases. “Treating” or “treatment” as used herein includes prevention of a condition or disease, and accordingly, prophylactic uses of the liposomal compositions of the invention are also included within the scope of the invention.

By a “cancer” or “neoplasm” is meant any unwanted growth of cells serving no physiological function. In general, a cell of a neoplasm has been released from its normal cell division control, i.e., a cell whose growth is not regulated by the ordinary biochemical and physical influences in the cellular environment. In most cases, a neoplastic cell proliferates to form a clone of cells which are either benign or malignant. Examples of cancers or neoplasms include, without limitation, transformed and immortalized cells, tumours, and carcinomas such as breast cell carcinomas and prostate carcinomas. The term cancer includes cell growths that are technically benign but which carry the risk of becoming malignant. By “malignancy” is meant an abnormal growth of any cell type or tissue. The term malignancy includes cell growths that are technically benign but which carry the risk of becoming malignant. This term also includes any cancer, carcinoma, neoplasm, neoplasia, or tumor.

Most cancers fall within three broad histological classifications: carcinomas, which are the predominant cancers and are cancers of epithelial cells or cells covering the external or internal surfaces of organs, glands, or other body structures (e.g., skin, uterus, lung, breast, prostate, stomach, bowel), and which tend to mestastasize; sarcomas, which are derived from connective or supportive tissue (e.g., bone, cartilage, tendons, ligaments, fat, muscle); and hematologic tumors, which are derived from bone marrow and lymphatic tissue. Carcinomas may be adenocarcinomas (which generally develop in organs or glands capable of secretion, such as breast, lung, colon, prostate or bladder) or may be squamous cell carcinomas (which originate in the squamous epithelium and generally develop in most areas of the body). Sarcomas may be osteosarcomas or osteogenic sarcomas (bone), chondrosarcomas (cartilage), leiomyosarcomas (smooth muscle), rhabdomyosarcomas (skeletal muscle), mesothelial sarcomas or mesotheliomas (membranous lining of body cavities), fibrosarcomas (fibrous tissue), angiosarcomas or hemangioendotheliomas (blood vessels), liposarcomas (adipose tissue), gliomas or astrocytomas (neurogenic connective tissue found in the brain), myxosarcomas (primitive embryonic connective tissue), or mesenchymous or mixed mesodermal tumors (mixed connective tissue types). Hematologic tumors may be myelomas, which originate in the plasma cells of bone marrow; leukemias which may be “liquid cancers” and are cancers of the bone marrow and may be myelogenous or granulocytic leukemia (myeloid and granulocytic white blood cells), lymphatic, lymphocytic, or lymphoblastic leukemias (lymphoid and lymphocytic blood cells) or polycythemia vera or erythremia (various blood cell products, but with red cells predominating); or lymphomas, which may be solid tumors and which develop in the glands or nodes of the lymphatic system, and which may be Hodgkin or Non-Hodgkin lymphomas. In addition, mixed type cancers, such as adenosquamous carcinomas, mixed mesodermal tumors, carcinosarcomas, or teratocarcinomas also exist.

Cancers may also be named based on the organ in which they originate i.e., the “primary site,” for example, cancer of the breast, brain, lung, liver, skin, prostate, testicle, bladder, colon and rectum, cervix, uterus, etc. This naming persists even if the cancer metastasizes to another part of the body, that is different from the primary site. Cancers named based on primary site may be correlated with histological classifications. For example, lung cancers are generally small cell lung cancers or non-small cell lung cancers, which may be squamous cell carcinoma, adenocarcinoma, or large cell carcinoma; skin cancers are generally basal cell cancers, squamous cell cancers, or melanomas. Lymphomas may arise in the lymph nodes associated with the head, neck and chest, as well as in the abdominal lymph nodes or in the axillary or inguinal lymph nodes. The following list provides some non-limiting examples of primary cancers and their common sites for secondary spread (metastases):

| Primary cancer | Common sites for metastases |
|---|---|
| prostate | bone |
| breast | bone, lungs, skin, brain |
| lung | bone, brain |
| colon | liver, lungs, bone |
| kidney | lungs, bone |
| pancreas | liver, lungs, bone |
| melanoma | lungs |
| uterus | lungs, bones, ovaries |
| ovary | liver, lung |
| bladder | bone, lung |

Tumor vasculature is generally leakier than normal vasculature due to fenestrations or gaps in the endothelia. This may allow liposomes of about 200 nm in diameter or less to penetrate the discontinuous endothelial cell layer and underlying basement membrane surrounding the vessels supplying blood to a tumor. Selective accumulation of the delivery vehicles into tumor sites following extravasation leads to enhanced delivery and effectiveness of the therapeutic agent. In order to promote extravasation, targeting agents directed against tumor associated endothelial cells may be bound to the outer surface of the liposomes. In some embodiments, a targeting antibody may be covalently or non-covalently incorporated on the surface of the liposome to enable specific localization of the liposome to areas of disease; for example metastatic cancer cells which have spread to other sites in the body. In some embodiments, a therapeutic antibody may be incorporated into the liposome.

Any therapeutic agent (e.g., a poorly soluble agent) may be formulated in the liposomal compositions of the invention. Suitable therapeutic agents for use according to the methods of the invention include, without limitation, azole compounds, such as econazole, miconazole, and clotrimazole. Suitable therapeutic agents also include drugs such as Taxol® (paclitaxel), an etoposide-compound (etoposide and derivatives of etoposide with a similar core structure including teniposide), a camptothecin-compound (including topotecan, ironotecan, lurtotecan, 9-aminocamptothecin, 9-nitrocamptothecin and 10-hydroxycamptothecin, including salts thereof), a vinca-alkaloid or analog thereof, etc.

### Pharmaceutical & Veterinary Compositions, Dosages, and Administration

In some embodiments, the compositions of the invention are particularly useful for the delivery of poorly soluble compounds. Compounds or agents in the liposomal compositions of the invention can be provided alone or in combination with other compounds or agents (for example, nucleic acid molecules, small molecules, peptides, or peptide analogues), in the presence any pharmaceutically acceptable carrier, in a form suitable for administration to mammals, for example, humans, cattle, sheep, etc. In some embodiments, the compositions may include an adjuvant. In some embodiments, the liposomal compositions may include a targeting agent to localize or direct the liposomes to the region or tissue requiring exposure to therapeutic doses of the therapeutic agent. In some embodiments, the targeting agent may be an antibody or component that selectively recognizes a tumor or diseased cell or tissue. If desired, treatment with a liposomal composition according to the invention may be combined with more traditional and existing therapies for the condition to be treated. Compounds according to the invention may be provided chronically or intermittently. “Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Conventional pharmaceutical practice may be employed to provide suitable formulations or compositions to administer the compounds to subjects suffering from or at risk for cancer, fungal infection, etc. In some embodiments, the pharmaceutical compositions are administered parenterally, i.e. intraarticularly, intravenously, subcutaneously, or intramuscularly or via aerosol. Aerosol administration methods include intranasal and pulmonary administration. In some embodiments, the pharmaceutical compositions are administered intravenously, intramuscularly or intraperitoneally by a bolus injection. For example, see Rahman et al., U.S. Pat. No. 3,993,754; Sears, U.S. Pat. No. 4,145,410; Papahadjopoulos et al., U.S. Pat. No. 4,235,871; Schneider, U.S. Pat. No. 4,224,179; Lenk et al., U.S. Pat. No. 4,522,803; or Fountain et al., U.S. Pat. No. 4,588,578.

Methods well known in the art for making formulations are found in, for example, “Remington's Pharmaceutical Sciences” (19^{th} edition), ed. A. Gennaro, 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. In some embodiments, a liposomal composition according to the invention is not suitable for topical administration. In some embodiments, a liposomal composition according to the invention is particularly suitable for parenteral administration, e.g., by injection.

The liposomal compositions according to the invention are in general capable of delivering an effective amount of a compound to a cell in a subject. An “effective amount” of a compound according to the invention includes a therapeutically effective amount or a prophylactically effective amount. A “therapeutically effective amount” refers to an amount effective, at dosages and for periods of time necessary, to achieve the desired therapeutic result. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount. A preferred range for therapeutically or prophylactically effective amounts of a compound may be any integer from 0.1 nM-0.1M, 0.1 nM-0.05M, 0.05 nM-15 μM or 0.01 nM-10 μM.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in unit dose form for ease of administration and uniformity of dosage.

In the case of vaccine formulations, an immunogenically effective amount of a compound of the invention can be provided, alone or in combination with other compounds, with an immunological adjuvant, for example, Freund's incomplete adjuvant, dimethyldioctadecylammonium hydroxide, or aluminum hydroxide. The compound may also be linked with a carrier molecule, such as bovine serum albumin or keyhole limpet hemocyanin to enhance immunogenicity.

In general, compounds and compositions of the invention should be used without causing substantial toxicity. Toxicity of the compounds and compositions of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

The compositions may be administered to any suitable subject. As used herein, a subject may be a human, non-human primate, rat, mouse, cow, horse, pig, sheep, goat, dog, cat, etc. The subject may be a clinical patient, a clinical trial volunteer, an experimental animal, etc. The subject may be suspected of having or at risk for having a disorder, be diagnosed with a disorder or be a control subject that is confirmed to not have the specific disorder of interest.

### Kits

The liposomal compositions of the invention may be provided in a kit, together with instructions for use. The kit may include a first container including an agent solubilized in a micelle, a second container including a liposome of a desired composition, and instructions for mixing the contents of the first and second containers at a desired ratio to provide a liposomal composition containing the agent (i.e., to provide a loaded liposome).

In alternative embodiments, the kit may include a first container including an agent; a second container including a micelle-forming compound; and a third container including a liposome of the desired composition, together with instructions for combining the contents of the first and second containers to form a micelle loaded with the agent, and for combining the micelle with the contents of the third container to prepare a loaded liposome containing the agent.

In some embodiments, the kit may include a second agent to be loaded into the liposome using convention techniques, prior to combining the liposome with a micelle.

The kit components may be stored at suitable temperatures or forms, e.g., room temperature, refrigerated (e.g., 4° C.), frozen (e.g., −20° C.), cryopreserved, dehydrated, etc., for suitable lengths of time.

Various alternative embodiments and examples of the invention are described herein. These embodiments and examples are illustrative and should not be construed as limiting the scope of the invention.

### EXAMPLE 1

### Liposomal Formulation

### Materials

Econazole was purchased from Sigma-Aldrich (St. Louis, Mo. USA) as a nitrate salt powder. Dipalmitoyl phosphatidylcholine (DPPC), dimyristoyl phosphatidylcholine (DMPC) and distearoyl phosphatidylethanolamine-poly(ethylene glycol)₂₀₀₀ (DSPE-PEG) with an average PEG molecular weight of 2000 were purchased from Avanti Polar Lipids (Albaster, Ala.). Tritiated cholesteryl hexadecyl ether ([³H]-CHE) and [¹⁴C]-distearoyl phosphatidylethanolamine-poly(ethylene glycol) ([¹⁴C]-DSPE-PEG₂₀₀₀) were purchased from Perkin Elmer (Boston, Mass., USA). Whatman Nuclepore 200 nm, 100 nm or 80 nm filters were used in a 3 ml Lipex Extruder, all from Northern Lipids (Vancouver, B.C., Canada). Sephadex G-50 and Sepharose CL-4B size-exclusion chromatography beads were also purchased from Sigma. Other reagents were either from Sigma or Fisher Chemicals (Fairlawn, N.J., USA). All solvents were HPLC grade. Water was prepared by a reverse osmosis system (MilliQ) and filtered (0.22 μm) prior to use. Buffers were also filtered prior to use (0.22 μm).

### Econazole UV Spectrophotometric Assay

Econazole was dissolved in methanol up to a concentration of 25 mg/ml and a characteristic absorption peak was discovered in the ultraviolet range (λ=271 nm). Econazole experimental samples were quantified by comparison with a standard curve (r²≧0.995) with a linear range of 0.05-1.0 mg/ml. For liposomal econazole, the absorbance readings of empty liposomes were subtracted from liposomal econazole samples as background, and samples were typically diluted at 1:10 (v/v) in methanol (to clarity) prior to analysis to solubilize the liposomes and econazole.

### Liposome Preparation

Lipid constituents were weighed out in the desired mole to mole ratio and solubilized in chloroform. A nonexchangeable, nonmetabolized radioactive lipid tracer, [³H]-CHE (˜0.5 μCi/μmol) was added to the dissolved lipids for lipid quantitation post extrusion (Derksen, 1987). The lipid solution was dried to a thin film under N₂ gas, followed by hydration with HEPES-buffered saline (HBS: 25 mM HEPES, 150 mM NaCl, pH 7.2) at 50° C. for DPPC and 37° C. for DMPC for 1 h with frequent vortexing. Five cycles of freeze and thaw were then performed with liquid N₂ and a 37° C. waterbath. The sample was then extruded at 50° C. (DPPC) or 37° C. (DMPC) by passing the sample 10 times through two stacked polycarbonate filters of 200 nm pore size with a Lipex (Mayer 1986). Quasi-elastic light scattering (Nicomp 270, Particle Sizing Systems, Santa Barbara, Calif.) was used to determine mean diameter and particle size distribution of the liposomes and micelles (Table 1).

TABLE 1

| | | Mean diameter after |
|---|---|---|
| Liposomal Phospholipid | | addition of DSPE-PEG |
| Formulation | Mean diameter | micelles at 50° C. |
| DPPC | >>1 um | 152.4 ± 48.8 nm |
| DPPC/econazole | 143.5 ± 45.6 nm | 165.3 ± 69.2 nm |
| DMPC | 159.4 ± 38.3 nm | 160.2 ± 52.7 nm |
| DMPC/econazole | 141.2 ± 41.0 nm | 139.9 ± 46.3 nm |

Particle size determined by quasi-elastic light scattering of liposomes immediately after extrusion through 2×200 nm filters and after the addition of DSPE-PEG micelles. Data represent mean±SD (n=3 to 6 independent preparations).

### Liposomal Formulations

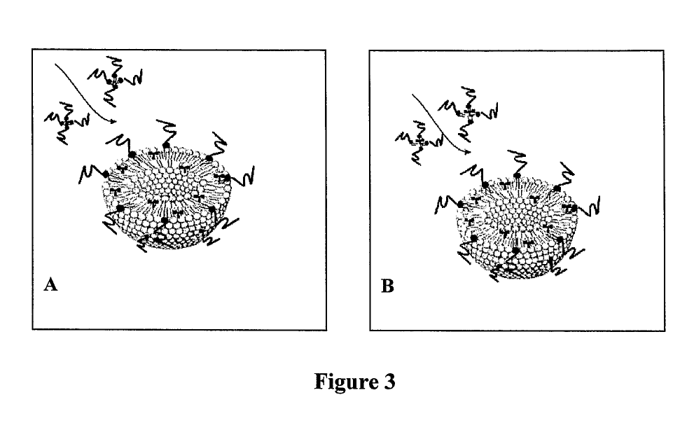

Econazole was incorporated into the lipid bilayer during liposome formation followed by exchange of DSPE-PEG₂₀₀₀ into the outer leaflet (FIG. 3A, thin-film/extrusion method), or econazole was incorporated into the outer leaflet of the lipid bilayer by exchange of DSPE-PEG/econazole micelles into pre-formed liposomes. (FIG. 3B, micelle-loading method) In the first case, DMPC or DPPC and econazole were mixed during the thin film stage of liposome preparation followed by extrusion, as described above. Separately, DSPE-PEG₂₀₀₀ was solubilized in HBS/ethanol (2:1 v/v), heated to 37° C. (DMPC) or 50° C. (DPPC) until clear micelles were formed (˜15 nm diameter) and then added to the warmed liposomes at 5 mol % and final ethanol 4.3% (v/v) (FIG. 3A). For the micelle-loading method, DMPC or DPPC liposomes were prepared first by extrusion as described above. Micelles of DSPE-PEG₂₀₀₀ in HBS/ethanol (2:1 v/v) also containing econazole were prepared by mixing and warming (50° C.) for approximately 30 min until clarity. By dynamic light scattering, the micelles formed were ˜15 nm diameter. Liposomes and micelles were then combined by mixing at 37° C. (DMPC) or 50° C. (DPPC) for the times indicated in the results section, up to 90 min. The final econazole concentration was 5 mg/mL and the lipid ratio (DMPC or DPPC: DSPE-PEG₂₀₀₀) was 95:5 (mol:mol). The ethanol concentration was 4.3% (v/v) upon first combining the liposomes and DSPE-PEG/econazole micelles.

### Analysis of Drug Loading

Liposomes were incubated with the micelles of DSPE-PEG±econazolefor 5, 15, 30, 60 or 90 minutes at 37° C. (DMPC-containing liposomes) or 50° C. (DPPC-containing liposomes). To separate liposome-associated econazole from free or micelle-associated econazole, 100 μl of sample were added with 50 μl of HBS in triplicate at each timepoint to 1 mL size exclusion Sephadex G-50 columns, and centrifuged at 792×g for 2 min. The minicolumns were pre-equilibrated in HBS (pH 7.2). The liposome-containing eluate was analyzed by UV spectroscopy for econazole as described above. Lipid concentration was measured by triplicate scintillation counting of the [³H]-CHE lipid tracer, and the drug:lipid ratio (w/w) was calculated at each timepoint. For each sample type, at least three independent liposome preparations were analyzed, and the mean drug:lipid ratio at each time point is reported.

### Stability Analyses

Stability testing was performed to observe how long the econazole would remain associated with the liposomes: a) in HBS at 4° C. and b) in the presence of human plasma at 37° C. For stability studies in buffer, liposomes were stored at 4° C. for 3, 10 or 20 days, then at each timepoint 100 μl of the sample were applied to mini Sephadex size exclusion columns in triplicate with 50 μl of HBS. The columns were centrifuged 792×g for 2 minutes and the elute was analyzed for lipid and econazole concentration as described above to determine the drug to lipid ratio.

### Results

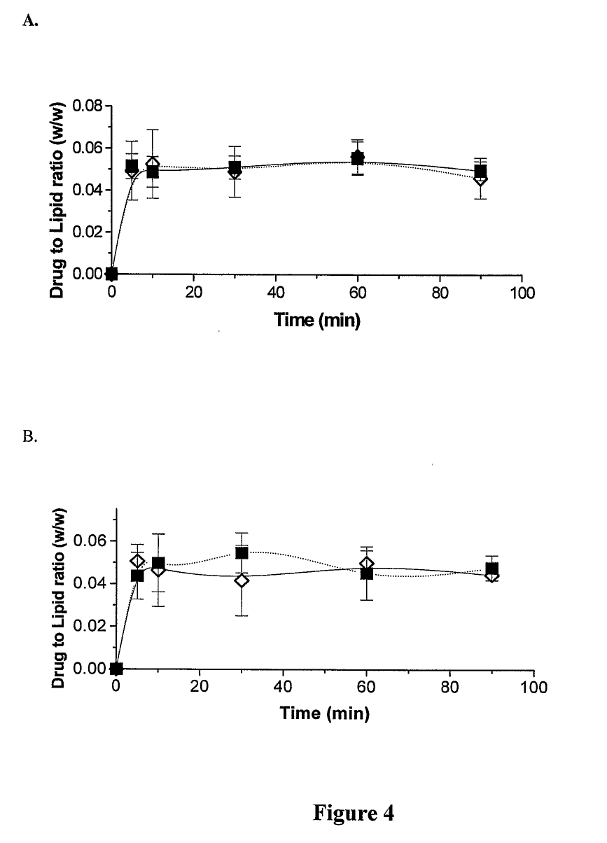

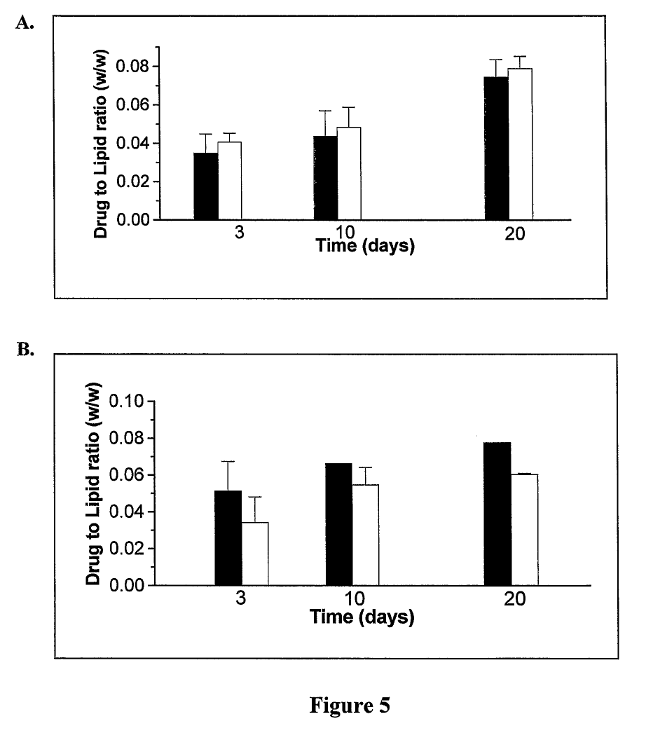

All liposomal formulations exhibited 100% drug loading at 0.05 drug:lipid ratio (w/w) of econazole (5 mg/mL). (FIG. 4) Significantly, the methods described here allowed for much easier hydration and extrusion steps than the formulations already containing PEG-lipid in the lipid film stage. Liposomes composed of 100% DPPC formed reversible aggregates quickly after extrusion. However, after the addition of DSPE-PEG₂₀₀₀ micelles and incubation at 50° C. for 30-60 min, a stable decrease in particle size and polydispersity indicated a reversal of the aggregation (Table 1). After the addition of DSPE-PEG₂₀₀₀ micelles with or without econazole the liposomal mean diameter was 140-160 nm. The lack of a significant separate particle population <50 nm is consistent with incorporation of the DSPE-PEG micelles or DSPE-PEG/econazole micelles into the liposomes. In the absence of PEG-lipid, control DPPC/econazole liposomes [100:5 (w/w)] were not stable and tended to aggregate within 2 hours. DMPC-based liposomes did not aggregate for any formulation step. Stability experiments showed that econazole remains stably associated with the liposomes for at least 3 weeks in HBS (pH 7.2) at 4° C. with no significant change from the 0.05 drug:lipid ratio originally loaded (FIG. 5) nor in particle size, with mean diameters remaining as in Table 1 throughout the study period.

### EXAMPLE 2

### Plasma Stability of Liposomal Econazole In Vitro

For stability studies in plasma, three separate preparations of micelle-loaded liposomal econazole were made with trace [¹⁴C]-CHE and [³H]-DSPE-PEG₂₀₀₀ as described above using DMPC or DPPC as the main lipid constituent. The liposomes were mixed with human plasma at a ratio of 1:3 (v/v) and incubated at 37° C. for 30 min. The plasma was applied to a 10 mL CL4B size exclusion chromatography column equilibrated in HBS and at least 25 fractions were collected at a rate of 0.7 ml/min to determine if econazole and PEG-lipid were associated with liposomes or with plasma protein-containing fractions. Each fraction was analyzed in triplicate for [¹⁴C]-CHE as a measure of the liposome-containing fractions, econazole, [³H]-DSPE-PEG₂₀₀₀ or total phosphate as a measure of PEG-lipid stability in the liposomes, and total protein. The three measures were averaged for each parameter, and these means were combined from the 3 different batches of liposomes for the data represented in the figures. Protein analysis was performed by visible spectrophotometry (λ=562 nm) using the bicinchoninic acid assay (Sigma) and compared to a triplicate standard curve of bovine serum albumin (linear range=0-100 μg/ml, r²≧0.995). The presence of empty liposomes, DSPE-PEG/econazole micelles or drug-loaded liposomes did not affect the fractional distribution of plasma proteins on the column. Likewise, the fractional distribution of the liposomes was not affected by the presence of econazole (in the liposomes or in DSPE-PEG micelles) or plasma proteins. Econazole analysis was by liquid-liquid extraction consisting of fraction sample, H₂O and ethyl acetate at a ratio of 1:1:6 (v/v/v). Samples were vortexed for 5 min and centrifuged at 10,000×g for 5 min. The top organic layer was removed, dried under N₂ gas and reconstituted in 100 μL methanol. The econazole assay was performed as described above. Background consisted of the corresponding extracted fractions of empty liposomes.

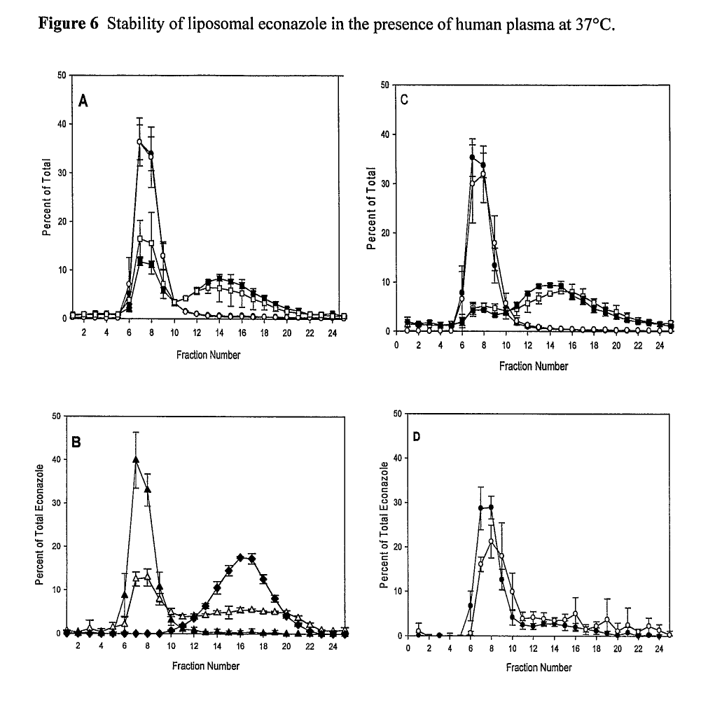

For the micelle-loaded liposomal econazole, stability in plasma was assessed by measuring drug:lipid ratio of the liposomes after incubation in plasma for 30 min at 37° C. Size exclusion chromatography was used to separate liposome-associated econazole from econazole associated with DSPE-PEG micelles or plasma proteins. For clarity of the figure, the liposome components and econazole are plotted in separate figures as percent of total component loaded onto the size exclusion columns. (FIG. 6) Approximately 49% of the econazole remained associated with the DPPC/DSPE-PEG liposomal fraction (FIG. 6, fractions 5-9) following the incubation period in plasma, compared to 95% for liposomes incubated in buffer. Approximately 34% was recovered in the partially overlapping protein and micelle fractions (FIG. 6, fractions 13-19) after incubation in plasma, compared to 2% in controls incubated in buffer. In the case of DMPC/DSPE-PEG/econazole micelle-loaded liposomes, 66% was recovered in the liposomal fraction after incubation in human plasma, compared to 81% in buffer, and 23% eluted in the protein/micelle fractions, compared to 13% eluting in those fractions after incubation in buffer. Due to the poor solubility of econazole in HBS (<0.1 mg/mL, near the limit of detection by the UV spectrophotometric assay) a separate free drug fraction was not detected upon elution from the column, but would likely have represented <1% of the total, based on mass balance of all collected fractions. Also of interest was the stability of the DSPE-PEG₂₀₀₀ in the liposomes. Approximately 37% of the DSPE-PEG was retained by the DPPC liposomal fraction after incubation in buffer and 50% after incubation in plasma, whereas in the DMPC-based liposomes, only 16% was retained in the liposomal fraction after incubation in buffer and only 20% after incubation in plasma. For this reason, the DPPC-based formulations were pursued in favor of the DMPC liposomes for the pharmacokinetic and efficacy studies, because the retention of PEG-lipid is presumed to be important in maximizing liposome circulation time and thereby tumor accumulation.

### EXAMPLE 3

### In Vivo Tolerability

### Multidose Tolerability Studies in Mice

Single dose and multi-dose tolerability studies were performed on Rag2M female mice at 50 mg/kg econazole dose via intravenous injection into the lateral tail vein at a volume of 200 μl/20 g mouse once (single dose) or every other day for 6 doses (multidose). The care, housing and use of animals were performed in accordance with the Canadian Council on Animal Care Guidelines. Four formulations were tested in the single-dose study, comparing DPPC and DMPC liposomes containing econazole prepared by the thin film/extrusion method vs. the micelle-loaded form. In all cases the final lipid ratio was 95:5 (mol/mol) (DPPC or DMPC: DSPE-PEG₂₀₀₀) and the drug:lipid ratio was 0.05 (w/w). The vehicle controls consisted of the corresponding liposomes not containing econazole. For the multidose study, only the DPPC-based liposomal econazole formulations were assessed prior to efficacy studies, because their stability was greater than the DMPC-based liposomes.

For both the single and multi-dose studies, mice (n=3/group) were weighed daily during the drug administration period and for 14 days after the last dose. Observation of appearance and behavior also continued for 14 days after the last dose and scored by a certified animal technician to ascertain morbidity. At the end of the study, the mice were terminated by CO₂ inhalation and blood was collected immediately by cardiac puncture. The blood was allowed to clot for 1 hour, and then the serum was separated by centrifugation 1000×g for 15 min. Serum was frozen in liquid N₂ and stored at −20 C until shipment to Central Laboratory for Veterinarians (Surrey, BC, Canada) for analysis of liver enzymes (alkaline phosphatase, AST, ALT, GGT, bilirubin, sorbital dehydrogenase), electrolytes, BUN and creatinine.

The single-dose tolerability study in Rag2M immunocompromised mice showed that the liposomal econazole formulations were all well tolerated at 50 mg/kg econazole dose [drug:lipid ratio=0.05 (w/w)] i.v. bolus with no obvious differences between treatment groups. The multidose tolerability study showed that DPPC-based liposomal econazole formulations were well tolerated at 50 mg/kg econazole [drug:lipid ratio=0.05 w/w)] i.v. bolus every other day excluding weekends for 6 doses. Serum was collected for analysis of liver enzymes (alkaline phosphatase, ALT, AST, GGT, bilirubin and sorbital dehydrogenase) in both the multidose tolerability study, at 14 days after the last of 6 doses, and in the efficacy study, at day 59 post tumor inoculation at the termination of the study (42 days after treatment stopped). In the multi-dose study, serum analysis indicated mild elevations in liver enzymes (ALT, GGT) in the liposomal econazole groups and less so in the vehicle control group (n=3 mice/group, lipid dose in all groups 1000 mg/kg) compared to the laboratory normal ranges for mice. (Table 2)

TABLE 2
Multidose tolerability of liposomal econazole

| | Alkaline | | ALT | AST | Bilirubin | |
|---|---|---|---|---|---|---|
| | phosphatase | GGT | (SGPT) | (SGOT) | (total) | |
| Treatment | (35-200) | (0-1) | (0-50) | (70-900) | (0-7) | Sorbital |
| group | IU/L | IU/L | IU/L | IU/L | μmol/L | Dehydrogenase |
| Empty | 160 ± 10 | 3.7 ± 1.2 | 79.3 ± 69.3 | 135 ± 65 | 5 ± 2 | 50.3 ± 37.3 |
| liposomes | | (↑) 3/3 | | | | |
| Liposomal | 149 ± 12.6 | 3 ± 1.7 | 60 ± 50.5 | 188 ± 88 | 4.3 ± 4.5 | 22.7 ± 13.1 |
| econazole, | | (↑) 2/3 | (↑) 1/3 | | | |
| conventional | | | | | | |
| Liposomal | 175 ± 7 | 3 ± 1 | 37.7 ± 6 | 129 ± 6.6 | 4 ± 2.6 | 26.3 ± 1.9 |
| econazole, | | (↑) 3/3 | | | | |
| micelle- | | | | | | |
| loaded | | | | | | |

Serum was collected 14 days after the last of 6 doses (50 mg/kg) i.v. every other day. Serum was pooled from 2 mice to produce 3 samples of sufficient volume for analysis (n=6 mice/group). Data represent mean±SD. Arrows indicate increase (t) above the normal range for mice, which is indicated at the top of each column.

Table 3 indicates the results of serum analysis from the efficacy study, where elevations in alkaline phosphatase, AST and GGT were noted, with greater increases associated with the liposomal econazole loaded by the thin film method. Alkaline phosphatase was elevated in all groups receiving liposomes, and in groups receiving econazole, bilirubin was slightly elevated in 1 of 3 samples in both groups. Results of serum electrolyte analysis showed elevated potassium levels in all groups receiving liposomes, however, BUN and creatinine were not elevated. (Table 4) Necropsy revealed pale liver and kidneys in several animals in all groups of the multidose study and the efficacy study, including the vehicle control group, which is consistent with the relatively high lipid dose.

TABLE 3
Liver enzyme changes in tumor-bearing mice that received liposomal econazole in an
efficacy study

| | | | | | | |
|---|---|---|---|---|---|---|
| Empty liposomes | 215 ± 34.6 | 4.7 ± 0.6 | 53 ± 11.7 | 122 ± 30 | 5.3 ± 1.5 | 33 ± 4.7 |
| | (↑) 2/3 | (↑) 3/3 | (↑) 2/3 | | | |
| Liposomal | 288 ± 90.7 | 3.7 ± 2.3 | 66 ± 7.6 | 192 ± 56 | 6 ± 4 | 32.6 ± 7.3 |
| econazole, | (↑) 2/3 | (↑) 2/3 | (↑) 3/3 | | (↑) 1/3 | |
| conventional | | | | | | |
| Liposomal | 225 ± 105 | 3.3 ± 3.0 | 36 ± 8.0 | 121 ± 46 | 8.3 ± 0.5 | 25.9 ± 1.6 |
| econazole, | (↑) 1/3 | (↑) 2/3 | | | (↑) 1/3 | |
| micelle-loaded | | | | | | |

Serum was collected at day 59 post-tumor inoculation from mice bearing MCF-7 xenograft tumors. Treatment with liposomal econazole (50 mg/kg i.v. for 6 doses) occurred on days 17, 20, 22, 24, 27 and 29). Serum was pooled from 2 mice to produce 3 samples of sufficient volume for analysis (n=6 mice/group). Data represent mean±SD. Arrows indicate increase (T) above normal range for mice, with the number of mice exhibiting the change indicated (e.g. 2 out of 3 samples: 2/3). Normal ranges for mice are indicated at the top of each column in parentheses.

TABLE 4
Serum electrolytes and renal function assessment in in tumor-bearing mice that
received liposomal econazole in an efficacy study

| | Na⁺ | K⁺ | Ca²⁺ | Phosphorus | Cl⁻ | CO₂ | BUN | Creatinine |
|---|---|---|---|---|---|---|---|---|
| Treatment | (143-152) | (0-1) | (2.14-2.54) | (1.73-3.51) | (103-117) | (14-28) | (6-17) | (30-56) |
| group | mmol/L | mmol/L | mmol/L | mmol/L | mmol/L | mmol/L | mmol/L | μmol/L |
| Empty | 148 ± 2 | 8.0 ± 0.3 | 2.5 ± 0.03 | 2.5 ± 0.1 | 113 ± 1 | 27.3 ± 1.2 | 6.6 ± 0.5 | 20 ± 3 |
| liposomes | | | | | | | | |
| Liposomal | 153 ± 5.8 | 7.9 ± 0.4 | 2.7 ± 0.2 | 2.4 ± 0.3 | 118 ± 5.9 | 27 ± 1.7 | 8.1 ± 3.8 | 23.3 ± 7.0 |
| econazole, | | | | | | | | |
| conventional | | | | | | | | |
| Liposomal | 150 ± 1.5 | 8.3 ± 0.5 | 2.6 ± 0.1 | 2.5 ± 0.1 | 115 ± 1.5 | 28.3 ± 1.5 | 6.1 ± 0.4 | 22.7 ± 5.0 |
| econazole, | | | | | | | | |
| micelle- | | | | | | | | |
| loaded | | | | | | | | |

Serum was collected at day 59 post-tumor inoculation from mice bearing MCF-7 xenograft tumors. Treatment with liposomal econazole (50 mg/kg i.v. for 6 doses) occurred on days 17, 20, 22, 24, 27 and 29). Serum was pooled from 2 mice to produce 3 samples of sufficient volume for analysis (n=6 mice/group). Data represent mean±SD. Arrows indicate increase (↑) above normal range for mice, which is indicated at the top of each column in parentheses.

### EXAMPLE 4

### Pharmacokinetics of Liposomal Econazole

Reverse-Phase HPLC Assay—For analysis of pharmacokinetic samples, 200 μl of plasma were extracted 2 times with 3 volumes of ethyl acetate and 2 volumes of 0.1M NaOH, with vortexing for 15 min for each extraction, and centrifugation at 1500×g for 10 min to separate organic and aqueous phases. The combined organic phases were dried at 60° C. under vacuum in a vortex-evaporator in approximately 20 min. The dried extract was reconstituted in 100-200 μL acetonitrile and centrifuged to remove any residue. The supernatant (10 μL) was injected onto the HPLC by autoinjector the same day. The HPLC column was a NovaPak RP-18 (C18, 75×46 mm, 4 μm) and the mobile phase was acetonitrile: 10 mM ammonium formate+20 mM diethylamine (64:36) run at a flow rate of 1 mL/min at 28° C. column temperature. UV detection (λ=270 nm) was performed with a photodiode array detector (Waters 996). Quantitation of samples was performed using an external standard curve of econazole prepared in triplicate in mouse plasma, using the same extraction method as the samples (r²>0.995, linear range: 20-250 μg/mL, limit of detection=10 μg/mL). Extraction efficiency, was ˜90% across the concentration range. Data analysis was performed using WinNonLin version 1.5 software (Scientific Consulting, Inc.,) and comparison of means was performed using MicroCal Origin software with two-way Anova, where significance was set at p=0.05.

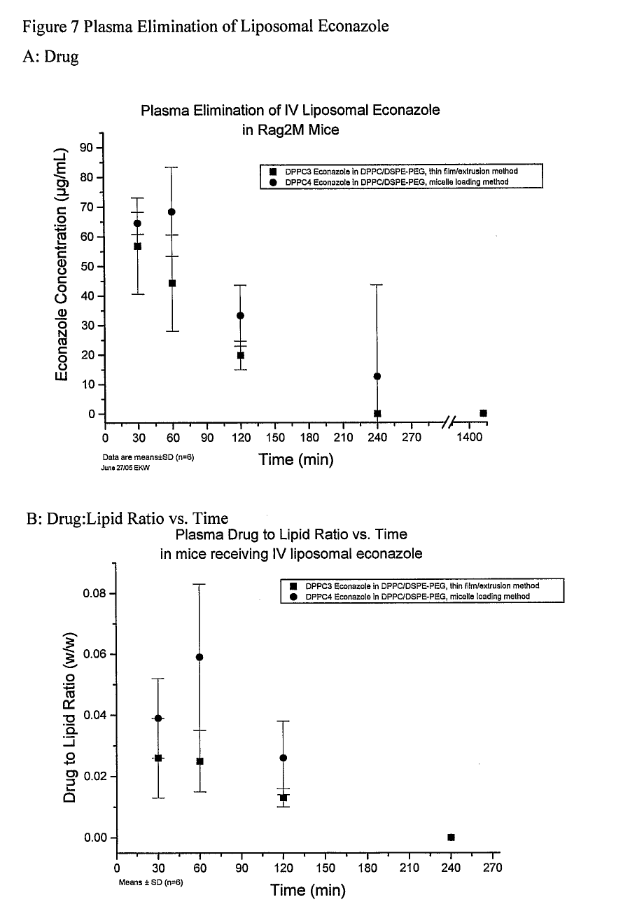

Rag2M mice were injected intravenously with liposomal econazole that was prepared by either the thin-film/extrusion method or by the micelle-loading method. Analysis of econazole concentration in the plasma vs. time (FIG. 7) showed that the majority of both formulations of liposomal econazole was cleared from the plasma by 2 hours and that elimination appears to follow a first-order elimination process. The area under the curve for the measured timepoints (AUC_{0-240 min}) was estimated to be 196 mg/ml·min for the thin-film/extrusion liposomal econazole and 281 mg/mL·min for the micelle-loaded liposomal econazole and plasma half-life of approximately 30.9 min, and 34.3 min, respectively. The drug-to-lipid ratio was significantly different between the two formulations at 15, 30 and 60 minutes (p<0.05), with the micelle-loaded form showing a higher drug-to-lipid ratio at those timepoints.

### EXAMPLE 5

### Efficacy of Liposomal Econazole in MCF-7 Xenografts in Rag2M Mice

Mice received estradiol as 60-day slow-release subcutaneous pellets one day prior to tumour cell inoculation. The mice were injected with 1×10⁵ MCF-7 cells (American Type Culture Collection, ATCC) subcutaneously. The mice were injected with 200 μl/20 g of liposomal econazole or empty liposomes via the lateral tail vein once the tumours reached approximately 50 mm³, with dosing every other day excluding weekends for a total of 6 doses, starting at day 17 post-tumor inoculation. Tumors were measured daily until day 59, at which time the mice were sacrificed and serum was collected for analysis as described above. Observation of appearance and behavior also continued throughout the study period, scored by a certified animal technician to ascertain morbidity.

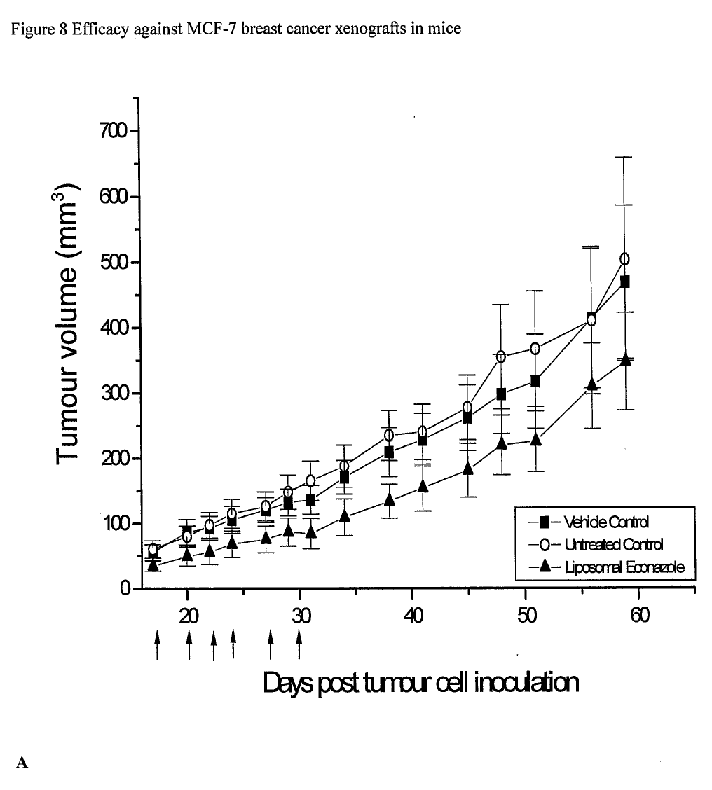

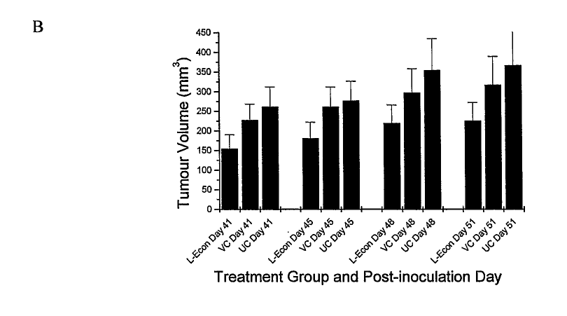

Liposomal econazole prepared using DPPC/DSPE-PEG₂₀₀₀ (95:5 mol/mol) by the micelle-loading and the thin-film/extrusion methods were chosen for in vivo testing because stability studies up to that point indicated that they would be more suitable than the DMPC-based formulations. Untreated controls and mice receiving empty liposomes (vehicle control) reached a tumor volume of 300 mm³ by day 48, whereas there was a 10-12 day tumor growth delay in the liposomal econazole groups. Mean±SEM There was also a trend to reduced tumor volume growth of the liposomal econazole groups, which behaved similarly, was significantly less than compared to that of the vehicle control group and untreated control group (Anova, p<0.05) (FIG. 8). It should be noted that tumor growth was relatively controlled in the liposomal econazole group, resulting in a lower tumor volume two days after completion of the 6 doses (Day 31) compared to the control groups (FIG. 8 inset), and the growth rate did not increase until treatment stopped.

### REFERENCES

The following publications are incorporated by reference:
E. Porges, B. Schade, W. Ropte, Automated flow-through method to determine the dissolution rate of slightly soluble substances, Pharm. Ind. 47(1) (1985) 77-86.
A. Gamberucci, R. Fulceri, A. Benedetti, F. L. Bygrave, On the mechanism of action of econazole, the capacitative calcium inflow blocker, Biochem. Biophys. Res. Comm. 248 (1998) 75-77.
J. Soboloff, S. A. Berger, Sustained ER Ca²⁺ depletion suppresses protein synthesis and induces activation-enhanced cell death in mast cells, J. Biol. Chem. 277(16) (2002) 13812-13820.
A. Najid, M. H. Ratinaud, Comparative studies of steroidogenesis inhibitors (econazole, ketoconazole) on human breast cancer MCF-7 cell proliferation by growth experiments, thymidine incorporation and flow cytometric DNA analysis, Tumori. 77(5) (1991) 385-390.
Y. Zhang, M. Crump, S. A. Berger, Purging of contaminating breast cancer cells from hematopoietic progenitor cell preparations using activation enhanced cell death, Breast Cancer Res Treatment. 72 (2002) 265-278.
J. Soboloff, Y. Zhang, M. Minden, S. A. Berger, Sensitivity of myeloid leukemia cells to calcium influx blockade: Application to bone marrow purging, Exp. Hem. 30 (2002) 1219-1226.
Y. Zhang, S. A. Berger, Increased calcium influx and ribosomal content correlate with resistance to endoplasmic reticulum stress-induced cell death in mutant leukemia cell lines, J. Biol. Chem. 279(8) (2004) 6507-6516.
A. Gabizon, D. C. Price, J. Huberty, R. S. Bresalier, D. Papahadjopoulos, Effect of liposome composition and other factors on the targeting of liposomes to experimental tumors: biodistribution and imaging studies, Cancer Res. 50(19) (1990) 6371-6378.
H. Maeda, J. Wu, T. Sawa, Y. Matsumura, K. Hori, Tumor vacular permeability and the EPR effect in macromolecular therapeutics: a review, J. Control. Rel. 65 (2000) 271-284.
T. Tanaka, S. Shiramoto, M. Miyashita, Y. Fujishima, Y. Kaneo, Tumor targeting based on the effect of enhanced permeability and retention (EPR) and the mechanism of receptor-mediated endocytosis (RME), I. J. Pharm. 277 (2004) 39-61.
T. D. Madden, P. R. Harrigan, L. C. Tai, M. B. Bally, L. D. Mayer, T. E. Redelmeier, H. C. Loughrey, C. P. Tilcock, L. W. Reinish, P. R. Cullis, The accumulation of drugs within large unilamellar vesicles exhibiting a proton gradient: a survey. Chem Phys Lipids, 53(1) (1990) 37-46.
B. Ashok, L. Arleth, R. P. Hjelm, I. Rubinstein, H. Onyuksel, In vitro characterization of PEGylated phospholipid micelles for improved drug solubilization: effects of PEG chain length and PC incorporation, J. Pharm. Sci. 93(10) (2004) 2476-2487.
A. L. Klibanov, K. Maruyama, V. P. Torchilin, L. Huang L., Amphipathic polyethyleneglycols effectively prolong the circulation time of liposomes, FEBS Lett., 268(1) (1990) 235-237.
D. Bhadra, S. Bhadra, P. Jain, N. K. Jain, Pegnology: a review of PEG-ylated systems, Pharmazie. 57(1) (2002) 5-29.
T. M. Allen, C. Hansen, F. Martin, C. Redemann, A. Yau-Young. Liposomes containing synthetic lipid derivatives of poly(ethylene glycol) show prolonged circulation half-lives in vivo, Biochim. Biophys. Acta, 1066(1) (1991) 29-36.
R. Homan, H. J. Pownall, Transbilayer diffusion of phospholipids: dependence on headgroup structure and acyl chain length, Biochim. Biophys. Acta, 938(2) (1988) 155-166.
H. Komatsu and S. Okada, Ethanol-enhanced permeation of phosphatidylcholine/phosphatidylethanolamine mixed liposomal membranes due to ethanol-induced lateral phase separation, Biochim. Biophys. Acta. 1283(1) (1996) 73-79.
H. V. Ly and M. L. Longo, The influence of short-chain alcohols on interfacial tension, mechanical properties, area/molecule, and permeability of fluid lipid bilayers. Biophys J. 87(2) (2004) 1013-1033.
Schott H Hydrophilic-lipophilic balance, solubility parameter, and oil-water partition coefficient as universal parameters of nonionic surfactants. J Pharm Sci. (1995) 84(10):1215-1222.
Schott H. Solubility parameter and hydrophilic-lipophilic balance of nonionic surfactants. J Pharm Sci. (1984);73(6):790-792
Adlakha-Hutcheon G, Bally M B, Shew C R, Madden T D. Controlled destabilization of a liposomal drug delivery system enhances mitoxantrone antitumor activity. Nat. Biotechnol. (1999) 17(8):775-779.
N. Dos Santos, K. A. Cox, C. A. McKenzie, F. van Baarda, R. C. Gallagher, G. Karlsson, K. Edwards, L. D. Mayer, C. Allen, M. B. Bally, pH gradient loading of anthracyclines into cholesterol-free liposomes: enhancing drug loading rates through use of ethanol, Biochim. Biophys. Acta. 1661(1) (2004) 47-60.
E. K. Wasan, S. Berger, S. Cogswell, M. Bally, United States Provisional patent: Method of liposomal formulation of hydrophobic compounds USPTO # is U.S. 60/647,419 January, 2005
S. Cogswell, S. Berger, F. Kuan, M. Bally, E. K. Wasan, Liposomal formulation of the hydrophobic drug econazole, a novel anticancer compound, via PEG-lipid mixed micelles and ethanol permeation. J Controlled Release (2005) annual meeting abstract, in press.
T. Derksen, H. W. Morselt, G. L. Scherphof, Processing of different liposome markers after in vitro uptake of immunoglobulin-coated liposomes by rat liver macrophages. Biochim. Biophys. Acta. 931(1) (1987) 33-40.
L. D. Mayer, M. J. Hope, P. R. Cullis, Vesicles of variable sizes produced by a rapid extrusion procedure, Biochim. Biophys. Acta. 858(1) (1986) 161-168
H. Van den Bossche, J. M. Ruysschaert, F. Difrise-Quertain, G. Willemsens, F. Cornelissen, P. Marichal, W. Cools, J. Van Cutsem. The interaction of miconazole and ketoconazole with lipids. Biochem. Pharmacol. 31(16) (1982) 2609-2617.

### OTHER EMBODIMENTS

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications are incorporated herein by reference as if each individual publication were specifically and individually indicated to be incorporated by reference herein and as though fully set forth herein. The invention includes all embodiments and variations substantially as hereinbefore described and with reference to the examples and drawings.

What is claimed is:

1. A method for loading an agent into a liposome, the method comprising:
a) combining the agent with a micelle-forming compound to form a micelle comprising the agent, wherein the agent is releasable from said micelle-forming compound; and
b) adding the micelle to the liposome, wherein the micelle combines with the liposome such that the agent is loaded into the liposome to form a loaded liposome.

2. The method of claim 1 wherein in step (b), the micelle combines with the lipid bilayer of the liposome.

3. The method of claim 1 or 2 wherein the micelle-forming compound comprises a hydrophilic or amphipathic moiety.

4. The method of claim 3 wherein the micelle-forming compound is a PEG-lipid conjugate.

5. The method of claim 4 wherein the PEG-lipid conjugate is DSPE-PEG2000.

6. The method of any one of claims 1 to 5 wherein the agent is dissolved in a solvent.

7. The method of claim 6 wherein the solvent is ethanol.

8. The method of any one of claims 1 to 7 wherein the agent is a compound that is poorly soluble.

9. The method of any one of claims 1 to 8 wherein the agent is a therapeutic agent.

10. The method of any one of claims 1 to 9 wherein the agent is econazole.

11. The method of any one of claims 1 to 9 wherein the agent is an anticancer agent or an antifungal agent.

12. The method of any one of claims 1 to 11 wherein the loaded liposome is about 100 nm to about 200 nm in diameter.

13. The method of any one of claims 1 to 12 wherein the loaded liposome is a unilamellar liposome.

14. The method of any one of claims 1 to 13 wherein the loaded liposome comprises one or more of a lipid selected from DMPC or DPPC.

15. The method of any one of claims 1 to 14 wherein the loaded liposome comprises a targeting agent.

16. A composition produced by the method of any one of claims 1 to 15.

17. The composition of claim 16 further comprising a pharmaceutically acceptable carrier.

18. A liposomal composition comprising econazole, wherein the composition is formulated for parenteral delivery.

19. The composition of claim 18 wherein the composition comprises a lipid selected from DMPC or DPPC.

20. The composition of claim 18 or 19 wherein the composition comprises DSPE-PEG2000.

21. A method of treating a cancer or a fungal infection comprising administering the composition of any one of claims 16 to 20 to a subject in need thereof.

22. Use of the composition of any one of claims 16 to 20 for preparation of a medicament for treating a cancer or a fungal infection in a subject in need thereof.

23. A method of delivering a therapeutic agent to a cell in a subject in need thereof comprising administering the composition of any one of claims 16 to 20 to said subject.

24. A method for selecting a liposome composition having a desired loading or retention property for an agent, the method comprising:
a) preparing a first liposome composition by combining a vesicle-forming lipid with the agent under conditions suitable for forming a liposome such that the agent is loaded into the liposome;
b) preparing a second liposome composition by combining the agent with a micelle-forming compound to form a micelle comprising the agent, wherein the agent is releasable from said micelle-forming compound, and adding the micelle to a liposome, wherein the micelle combines with the liposome such that the agent is loaded into the liposome;
c) determining the amount of agent loaded onto the liposome or retained in the liposome in the first liposome composition and the second liposome composition,
wherein a greater amount of agent loaded onto the liposome or retained in the liposome in the second liposome composition indicates a liposome composition having a desired loading or retention property in vitro or in vivo for the agent.

25. A kit for preparing a loaded liposome comprising a first container comprising an agent solubilized in a micelle and a second container comprising a liposome of the desired composition, together with instructions for combining the contents of the first and second containers to prepare a loaded liposome.

26. A kit for preparing a loaded liposome comprising a first container comprising an agent; a second container comprising a micelle-forming compound; and a third container comprising a liposome of the desired composition, together with instructions for combining the contents of the first and second containers to form a micelle comprising the agent, and for combining the micelle with the contents of the third container to prepare a loaded liposome.

27. The kit of claim 25 or 26 wherein the agent is a therapeutic agent.

28. The kit of any one of claims 25 to 27 wherein the therapeutic agent is econazole.

29. The kit of any one of claims 25 to 28 wherein the micelle comprises DSPE-PEG2000.

30. The kit of any one of claims 25 to 29 wherein the liposome comprises a lipid selected from DMPC or DPPC.
